(19) United States
(12) Patent Application Publication
(10) Pub. No.: US 2014/0294668 A1
(43) Pub. Date: Oct. 2, 2014

(54) LIGHT BEAM DISINFECTION DEVICE AND METHOD THEREOF

(71) Applicant: Kun-Ta LEE, New Taipei City (TW)

(72) Inventor: Kun-Ta LEE, New Taipei City (TW)

(21) Appl. No.: 14/224,563

(22) Filed: Mar. 25, 2014

(30) Foreign Application Priority Data
Mar. 28, 2013 (TW) 102111260

(51) Int. Cl. A61L 2/08; A61L 2/10

(52) U.S. Cl. CPC A61L 2/08; A61L 2/10
USPC 422/22; 250/492.1; 422/24

(57) ABSTRACT

A light beam disinfection device and a method thereof are provided. The light beam disinfection device comprises a housing, a driven system, a power generating device and a light source. The housing covers a handrail. The driven system is disposed in the housing and comes into contact with the handrail. The power generating device connects with the driven system and generates electric power while the driven system is rolling. The light source is disposed in the housing and provides a light beam when receiving the electric power from the electrical power generating device. When the escalator is operating, the action of sliding the handrail is adapted to drive the driven system. The power generating device provides the electric power by rolling the driven system, and thus, the light source provides the light beam for the handrail of the escalator.

This application claims the benefit from the priority to Taiwan Patent Application No. 102111260 filed on Mar. 28, 2013, the disclosures of which are incorporated by reference herein in their entirety.

### CROSS-REFERENCES TO RELATED APPLICATIONS

Not applicable.

### BACKGROUND OF THE INVENTION

1. Field of the Invention

The present invention relates to a light beam disinfection device and a method thereof.

In particular a light beam disinfection device for use in an escalator and a method thereof is provided.

2. Descriptions of the Related Art

With the improvement of living standards over recent years, public health issues have drawn attention, especially with highly infectious diseases such as SARS, enterovirus, H1N1 and noro virus. Frequent interactions between individuals through direct or indirect physical contact can easily spread infectious diseases. The most frequently touched items are escalators in public places.

Escalators in public places, e.g., the MRT stations or department stores, transport numerous people every day, so if anyone with the aforesaid diseases touches an escalator, the escalator is likely to become an ideal breeding ground for diseases and passes the diseases indirectly and rapidly to others.

Although escalator maintenance institutions maintain and disinfect the escalators at regular intervals according to regulations, the most commonly used disinfection method is to dilute alcohol with isopropanol or water and then use the mixed fluid to manually wipe the escalator. Unfortunately, this leaves a wet surface on the handrail and will also dry out users' skin.

Accordingly, an urgent need exists in the art to provide a disinfection device for use in an escalator and a method thereof.

### SUMMARY OF THE INVENTION

An objective of the present invention is to provide a light beam disinfection device which can be disposed on a handrail of an escalator to disinfect the handrail when the escalator is operating.

Another objective of the present invention is to provide a light beam disinfection method which can transform the kinetic energy of an escalator when the escalator is operating into electric energy to light up a light source. The light source can provide a light beam to the handrail of the escalator to perform the disinfection operation.

To achieve the aforesaid objectives, the light beam disinfection device disclosed in the present invention is disposed on the handrail of an escalator and comprises a housing, a driven system, a power generating device and a light source. The housing is adapted to cover the handrail; the driven system is disposed in the housing and in contact with the handrail; the power generating device is connected with the driven system to enable the light source to provide a light beam to the handrail for disinfection after the light source is disposed in the housing.

To achieve the aforesaid objectives, the present invention also discloses a light beam disinfection method for a handrail of an escalator. The method comprises the following steps: (a) providing a housing for covering the handrail; (b) providing a driven system disposed in the housing and in contact with the handrail; (c) providing a power generating device connected with the driven system and adapted to generate the electric power while the driven system is moving; and (d) providing a light source disposed in the housing, wherein the light source provides a light beam to the handrail for disinfection when receiving the electric power from the power generating device.

### BRIEF DESCRIPTION OF THE DRAWINGS

FIG. 1 is a schematic view of a light beam disinfection device disposed in an escalator according to the present invention;

FIG. 2 is a cross-sectional structural view of a light beam disinfection device according to the first embodiment of the present invention;

FIG. 3 is a cross-sectional structural view of a light beam disinfection device according to a second embodiment of the present invention; and

FIG. 4 is a flowchart diagram of a light beam disinfection method according to the present invention.

### DESCRIPTION OF THE PREFERRED EMBODIMENT

In the following description, the present invention will be explained with reference to embodiments thereof. However, the description of these embodiments is only for the purpose of describing the technical content and objectives of the present invention rather than to limit the present invention. It shall be appreciated that in the following embodiments and attached drawings, elements unrelated to the present invention are omitted from depiction. The numbers of, dimensions of and positional relationships among individual elements in the attached drawings are illustrated only for ease of understanding, but not to limit the actual numbers, scales and dimensions of the elements.

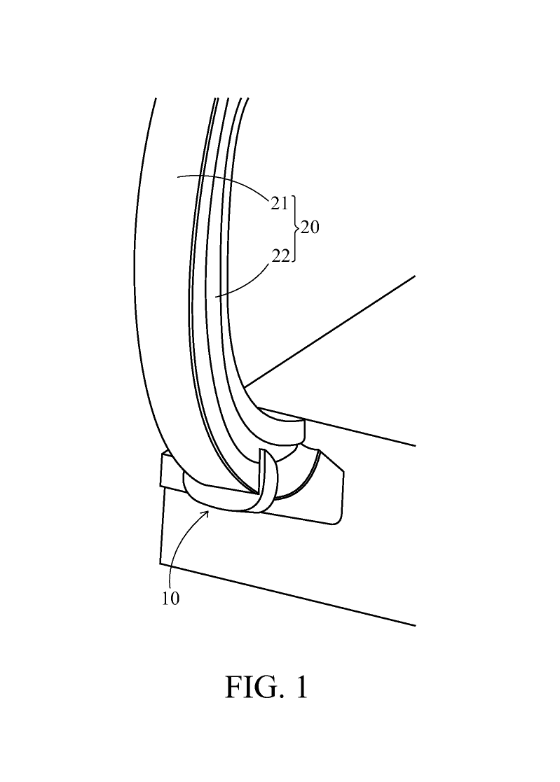

A schematic view illustrating the installation of a light beam disinfection device 10 of the present invention is shown in FIG. 1. The light beam disinfection device 10 is disposed on a handrail 21 of an escalator 20 and preferably covers the handrail 21 to adequately disinfect the handrail 21 of the escalator 20.

It shall be appreciated that as shown in FIG. 1, the light beam disinfection device 10 is preferably disposed at a position where the handrail 21 moves into or out of the escalator 20 to reduce the possible effects on the users of the escalator 20 due to the installation of the light beam disinfection device 10, but it is not limited thereto.

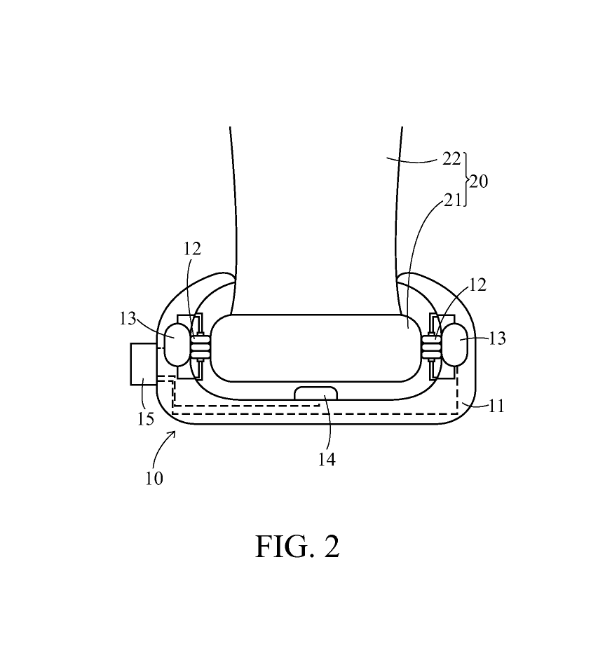

A first embodiment of a light beam disinfection device 10 according to the present invention is shown in FIG. 2. The light beam disinfection device 10 comprises a housing 11, a driven system 12, a power generating device 13 and a light source 14. As shown in FIG. 2, the housing 11 is adapted to cover the handrail 21 of the escalator 20 and may be designed in structure to be fixedly connected with the base 22 of the handrail 21. The driven system 12 is disposed in the housing 11 and is preferably composed of a roller system to come into contact with the handrail 21. The power generating device 13 is electrically connected with the driven system 12 and can generate power while the driven system 12 is rolling. The light source 14 is disposed in the housing 11 and adapted to provide a light beam to the handrail 21 when receiving the electric power from the power generating device 13.

In detail, in this embodiment, the housing 11 is preferred to have a U-shaped appearance to be tightly clamped to the base 22 of the escalator 20 and completely cover the handrail 21, but it is not limited thereto. In addition, a rubber material may be added to the connection portion between the housing 11 and the base 22 to stabilize the fixation and prevent the housing 11 from sliding along the handrail 21. The driven system 12 inside the housing 11 is disposed at one side of the handrail 21 and comes into contact with the side of the handrail 21. In this way, when the escalator 20 is operating, the action of sliding the handrail 21 is adapted to drive the driven system 12 to roll so that the power generating device 13 electrically connected with the driven system 12 generates electric power. The light source 14 is a fluorescent tube or a light-emitting diode, but it is not limited thereto. In addition to the single light source illustrated in this embodiment, a plurality of light sources may be connected in series or in parallel to provide light beams with appropriate and sufficient energy to disinfect the handrail 20. The light source 14 is preferred to be disposed inside the housing 11 to avoid the leakage of the light beam.

The aforesaid light beam for disinfection is a UV light beam, and specifically, a short-wave UVC light beam with a wavelength ranging between 100 nm and 280 nm. The UV light beam with a wavelength of 253.7 nm has the best effect for disinfection, but the present invention is not limited thereto.

The aforesaid way in which the electric power is generated by rolling the driven system 12 is not limited herein. That is, the rotational kinetic energy of the driven system 12 can be transformed into electric energy through electromotive force induction, electromagnetic transformation or by using a piezoelectric material. In addition, the light beam disinfection device 10 may further comprise a power storage device 15 which may be disposed outside the housing 11 or at some other position. The power storage device 15 is preferably a battery, which is adapted to save the electric power generated by the power generating device 13 and then supply the electric power to the light source 14 depending on the actual operations to improve the disinfection efficiency of the light beam disinfection device 10.

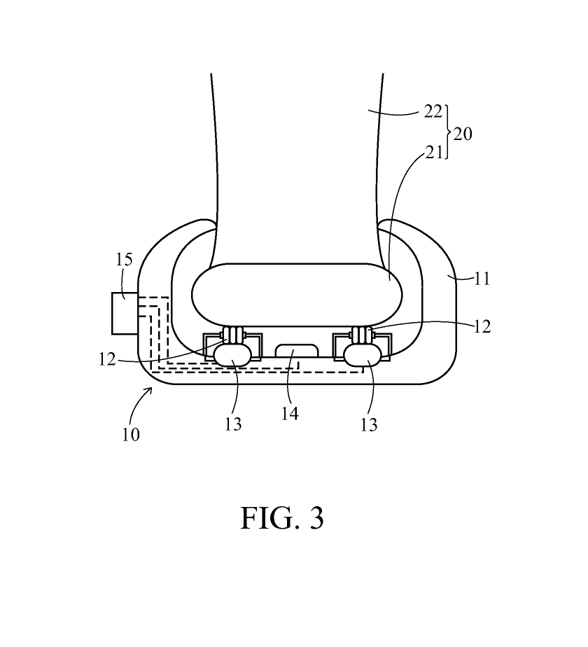

FIG. 3 illustrates a second embodiment of the light beam disinfection device 10 of the present invention. Similar to the first embodiment of FIG. 2, the light beam disinfection device 10 shown in FIG. 3 also operates in the following way: the power generating device 13 generates electric power while the driven system 12 is rolling so that the light source 14 can emit a light beam for disinfection. The second embodiment differs from the first embodiment in that the driven system 12 of the second embodiment is disposed relative to the upper surface of the handrail 21 and comes into contact with the upper surface of the handrail 21. In this way, when the escalator 20 is operating, the action of sliding the handrail 21 is adapted to drive the driven system 12 to roll so that the power generating device 13 electrically connected with the driven system 12 generates electric power.

The advantage of disposing the driven system 12 relative to the upper surface of the handrail 21 lies in that the number of elements of the driven system 12 can be increased if necessary. In this way, as compared with the first embodiment, more of the sliding kinetic energy of the handrail 21 can be transformed into electric energy when the handrail 21 slides at the same speed during the operation of the escalator 20.

If the surroundings of the escalator 20 are too messy or the composition of the users of the escalator 20 is too complicated, the light emitting power of the light source or the wavelength of the light beam may be adjusted according to the demands to effectively disinfect the handrail 21 of the escalator 20. On the other hand, the disinfection capability of the light beam disinfection device 10 may also be improved by cascading several light beam disinfection devices 10 to lengthen the time length during which the handrail 21 of the escalator 20 is irradiated.

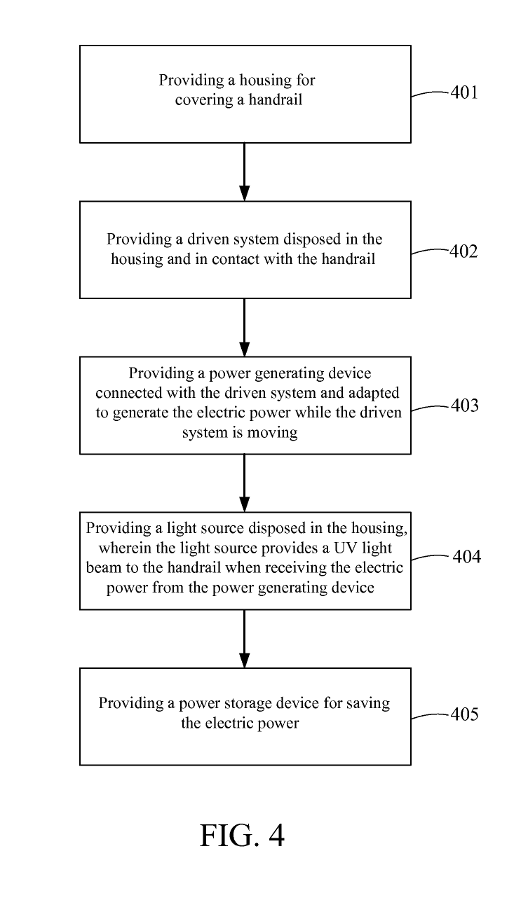

As shown in FIG. 4, a light beam disinfection method disclosed in the present invention comprises the following steps. Firstly, as shown in step 401, a housing 11 is provided for covering the handrail 21. Then, as shown in step 402, a driven system 12 is disposed in the housing 11 and comes into contact with the handrail 21. As shown in step 403, a power generating device 13 is provided to be connected with the driven system 12 to generate electric power while the driven system 12 is moving. Finally, as shown in step 404, a light source 14 is provided to be disposed in the housing 11 so that a light beam is provided to the handrail 21 by the light source 14 when receiving the electric power from the power generating device 13, thus, achieving the disinfection purpose of the present invention. For the unused electric power, a power storage device 15 may be provided for saving the electric power as shown in step 405.

According to the above descriptions, through the use of the light beam disinfection device and method of the present invention, the light source 14 can provide a light beam to the handrail 21 of the escalator 20 to disinfect the handrail 21 without the need of an additional power source. As a result, the costs for providing disinfection fluids, such as alcohol, and disruption to users' skin can be avoided. Moreover, the light beam disinfection device 10 of the present invention is independent from the escalator 20, so it is suspended on the escalator 20 as an additional device. In other words, the light beam disinfection device 10 of the present invention can be installed without changing the original structure of the escalator 20, which makes it convenient for the maintenance institutions to install the light beam disinfection device 10 to more effectively disinfect the handrail 21.

The above disclosure is related to the detailed technical contents and inventive features thereof People skilled in this field may proceed with a variety of modifications and replacements based on the disclosures and suggestions of the invention as described without departing from the characteristics thereof Nevertheless, although such modifications and replacements are not fully disclosed in the above descriptions, they have substantially been covered in the following claims as appended.

What is claimed is:

1. A light beam disinfection device, disposed on a handrail of an escalator, comprising:
a housing, adapted to cover the handrail;
a driven system, disposed in the housing and in contact with the handrail;
a power generating device, electrically connected with the driven system and adapted to generate electric power while the driven system is rolling; and
a light source, disposed in the housing and adapted to provide a light beam when receiving the electric power from the power generating device;
wherein when the escalator is operating, the sliding of the handrail is adapted to drive the driven system, the power generating device supplies the electric power by the rolling of the driven system, and thus the light source provides the light beam to the handrail of the escalator.

2. The light beam disinfection device according to claim 1, further comprising a power storage device for saving the electric power.

3. The light beam disinfection device according to claim 1, wherein the driven system is a roller system.

4. The light beam disinfection device according to claim 1, wherein the housing has a U-shaped appearance.

5. The light beam disinfection device according to claim 1, wherein the light beam is a UV light beam.

6. The light beam disinfection device according to claim 1, wherein the light source is a fluorescent tube or a light-emitting diode.

7. A light beam disinfection method for a handrail of an escalator, comprising:
(a) providing a housing for covering the handrail;
(b) providing a driven system disposed in the housing and in contact with the handrail;
(c) providing a power generating device electrically connected with the driven system and adapted to generate the electric power while the driven system is moving; and
(d) providing a light source disposed in the housing, wherein the light source provides a light beam to the handrail when receiving the electric power from the power generating device.

8. The method according to claim 7, further comprising:
(e) providing a power storage device for saving the electric power.

9. The method according to claim 7, wherein the driven system is a roller system.

10. The method according to claim 7, wherein the housing has a U-shaped appearance.

11. The method according to claim 7, wherein the light beam is a UV light beam.

12. The method according to claim 7, wherein the light source is a fluorescent tube or a light-emitting diode.
